(19) United States
(12) Patent Application Publication
(10) Pub. No.: US 2011/0301684 A1
(43) Pub. Date: Dec. 8, 2011

(54) SYSTEM AND METHOD FOR PERFORMING ANGIOGRAPHY AND STENTING

(75) Inventors: ROBERT E. FISCHELL, DAYTON, MD (US); DAVID R. FISCHELL, FAIR HAVEN, NJ (US); TIM A. FISCHELL, KALAMAZOO, MI (US)

(73) Assignee: SVELTE MEDICAL SYSTEMS, INC., NEW PROVIDENCE, NJ (US)

(21) Appl. No.: 12/795,707

(22) Filed: Jun. 8, 2010

(51) Int. Cl. A61M 25/00; A61F 2/84

(52) U.S. Cl. 623/1.11; 600/435

(57) ABSTRACT

An integrated catheter system for performing angiography on a human patient, the integrated catheter system consisting of an angiographic catheter onto which a thin-walled sheath is co-axially mounted. The angiographic catheter having an essentially straight and elongated proximal section in the form of a cylindrical shaft that is surrounded for less than one-half of its length by the thin-walled sheath that has an outer diameter that is less than 0.25 mm greater than the outside diameter of the angiography catheter. The strength to prevent buckling of the thin-walled sheath being provided by the shaft of the angiographic catheter. The integrated catheter system also being ideal for the placement of stents using small diameter stent delivery systems such as the stent-on-a-wire system.

### FIELD OF USE

This invention is in the field of means and methods for the treatment of stenoses that occur in human blood vessels by the implantation of intravascular stents.

### BACKGROUND OF THE INVENTION

At the present time in the history of stenting, stenoses of the arterial system are typically diagnosed with the use of angiography in which contrast medium is injected into the suspected vascular region and x-ray imaging is used to determine if there is a stenosis. To accomplish coronary angiography, the interventional cardiologist typically advances an angiographic catheter through an introducer sheath at the patient's groin with the distal end of the angiographic catheter being placed at the ostium of the coronary artery in the vascular region that is being examined. If a clinically significant stenosis is revealed by angiography, and if stenting is to be used as the means to dilate that stenosis, the angiographic catheter and (often) the introducer sheath are replaced with a new introducer sheath and guiding catheter both of which typically have a larger diameter as compared to the angiographic catheter and its introducer sheath. Typically, the catheter used for angiography will be sized from 4 FR to 6 FR (˜1.3 to 2 0 mm) in diameter and for stenting, the minimum diameter guiding catheter would typically be 6 FR (2 mm). The FR (French) diameter of a catheter expressed in millimeters is equal to the FR size divided by three. As an example, a 6 FR guiding catheter would have an outside diameter of 6/3=2 mm. A 6 FR introducer sheath used with a 6 FR guiding catheter would have to have an inside diameter that would allow the 6 FR guiding catheter to pass through which requires that the sheath have an even larger outside diameter which is typically 8 FR (˜2.7 mm). For coronary stenting, a 7 FR guiding catheter is often used and the associated introducer sheath would have a 9 FR (3 mm) outside diameter. With these sheaths with 2.7 mm to 3 mm diameters, it is not unusual to have bleeding at the groin after the introducer sheath is removed. That type of bleeding is potentially dangerous for the patient and occasionally requires a blood transfusion, vascular surgery or both to repair vascular damage. Such additional treatments are both expensive, time consuming and uncomfortable for the patient. In addition, major bleeding at the sheath insertion site is an independent predictor of late mortality after coronary artery stenting.

U.S. Pat. Nos. 5,180,376, 5,324,262 and 5,423,774 describe a non-kinking introducer sheath that includes a flat wire helix in the plastic of the sheath tube. The designs of these patents are generally greater in wall thickness than existing sheaths and do not envision the combination of an ultra-thin wall sheath with a combination angiography & guiding catheter.

U.S. Pat. No. 5,389,090 describes a unique guiding catheter that can be used to reduce the size of the opening made in the femoral artery when doing angiography and/or stenting. By using a dilator to place the guiding catheter through the femoral artery at the groin, the introducer sheath could possibly be eliminated thereby reducing by an additional two French sizes the size of the opening that is made in the femoral artery at the groin. Any reduction in the size of the opening in the groin is highly advantageous for reducing bleeding at that site in the femoral artery after the introducer sheath (if used) and the guiding catheter are removed. However, most interventional cardiologists prefer to have an introducer sheath left in the patient during the procedure to allow changes in catheter shapes, as well as when the procedure is over to inject medications and to allow quick access back into the artery. Therefore, a catheter system that could reduce the size of the opening in the groin while also having an introducer sheath for the angiographic catheter to slide through would be advantageous for the stenting or angioplasty of any coronary (or other) artery.

### SUMMARY OF THE INVENTION

When angiography is performed, there is a need for stenting or angioplasty in only about one third of such procedures. When stenting following angiography is not needed, it is still advantageous for the patient to have as small an opening made at the groin (or in the arm for radial artery stenting) as is possible. Therefore, the Intro-Angio-Guide catheter as described herein can be a very valuable tool for the interventional cardiologist whether or not angiography is or is not followed by stenting. This is true for doing angiography with or without stenting for all regions of a human body including peripheral arteries, bypass grafts and arteries of the heart, brain, kidneys, etc. The Intro-Angio-Guide catheter as described herein is ideally suited to be used with the stent-on-a-wire (S-O-A-W) stent delivery system as described in U.S. Pat. No. 6,375,660, or any other low profile balloon angioplasty or stent delivery system.

The present invention is called an Intro-Angio-Guide because it combines in one device; (1) an introducer sheath; (2) an angiographic catheter; and (3) a guiding catheter for use with a balloon angioplasty catheter, or a low profile stent delivery system such as the stent-on-a-wire (S-O-A-W) stent delivery system. Another way of looking at this device is that it combines, (1) an introducer sheath: and (2) an Angio-Guide catheter which can serve as an angiographic catheter or as a guiding catheter for a low profile stent delivery system, and (3) a sheathing catheter that is soft and flexible enough in its distal portion to allow it to be tracked over the shaft of a stent delivery system, such as S-O-A-W, to allow backup and low volume contrast injection in the more distal part of the coronary artery during stent placement. Thus, a single catheter (the Angio-Guide catheter) can serve as both an angiographic catheter to perform angiography and as a guiding catheter when using the S-O-A-W or another small diameter delivery system for the stenting of a blood vessel. The advantage of the present invention compared to existing devices is that the introducer sheath fits snugly over the cylindrical shaft of the Angio-Guide catheter and it has a wall thickness of less than 5 mils (a mil is 1/1,000 of an inch). It is practical to have a wall thickness for the introducer sheath of the Intro-Angio-Guide catheter that is only 2 to 3 mils. Because the thin-walled introducer sheath is inserted through the groin when wrapped around the Angio-Guide catheter, it does not buckle as it would, at that very thin wall thickness, if it weren't wrapped around a comparatively strong cylinder, namely, the shaft of the Angio-Guide catheter.

For coronary angiography, the following procedure is used after a guide wire has been placed through the groin and advanced through the femoral artery:
1. The dilator is inserted into the Intro-Angio-Guide catheter and the dilator and the Angio-Guide are advanced over the guide wire, through the skin and soft tissue, and via the femoral (or other access artery) to an area near the target vessel to be studied or treated.
2. After insertion of the system, the dilator and the guidewire are removed and the proximal end of the introducer sheath is secured to the skin with a suture.
3. The distal end of the Angio-Guide catheter is placed into the ostium of the target vessel (e.g., left coronary artery) where angiography is to be performed.
4. Contrast medium is injected at the proximal end of the Angio-Guide catheter and any stenosis is observed by x-ray imaging.
5. If no treatable stenosis is observed, the Intro-Angio-Guide is removed from the patient's body, but if a stenosis that requires angioplasty or stenting is identified, then a S-O-A-W stenting system or other low profile balloon angioplasty or stent system can be used to treat that stenosis, without requiring the exchange of the Intro-Angio-Guide to a new guiding catheter.

Because the introducer sheath fits snugly over the shaft of the Angio-Guide catheter with a clearance of only 1 to 2 mils, and because it has such a thin wall, the outside diameter of the Intro-Angio-Guide catheter will only be about 0.6 FR larger than the outside diameter of the Angio-Guide catheter. This compares with a diameter increase of about 2.0 FR when a conventional introducer sheath is used. This smaller outer diameter for the Intro-Angio-Guide system will tend to reduce the risk of bleeding at the groin that can be a serious problem for patients undergoing angiography or stenting. To facilitate the thinnest wall introducer sheath component with the maximum wall strength, a flat wire helix or braid may be included within the plastic of the present invention Intro-Angio-Guide introducer sheath. It is also envisioned that the distal end of the sheath, the distal end of the Angio-Guide catheter and the distal end of the straightening dilator used with the Angio-Guide catheter would have radiopaque markers to facilitate delivery under fluoroscopy. It is also envisioned that the distal 10-20 cm of the Angio-Guide will be relatively soft and flexible to allow it to be tracked down a coronary artery over the shaft of a balloon angioplasty catheter or a stent delivery system, and thus act as a distal support catheter, providing for superb “backup” to allow better steering and lesion crossing of the stent system as well as imaging of distal target lesions using a minimum of contrast.

The inventive means and method described herein is particularly valuable for the new type of stent delivery system (the S-O-A-W system) that is described in U.S. Pat. No. 6,375,660. The very small stent delivery system outside diameter made possible by the S-O-A-W system can allow, for the first time, the use of the same diameter introducer sheath and guiding catheter both for angiography and for stenting. Specifically, a 4 FR, 4.5 FR or 5 FR Intro-Angio-Guide catheter can be used to first perform angiography. If stenting of a stenosis is then required, the S-O-A-W stent delivery system can accomplish the stenting procedure using the same Intro-Angio-Guide system as was used for angiography. If a conventional stent delivery catheter is used to deliver a conventional stent, this would often require an introducer sheath and a guiding catheter with a larger diameter as compared to the Intro-Angio-Guide. It is anticipated that a unique Intro-Angio-Guide catheter whose size is 4.5±0.3 FR would be ideal for first performing angiography and then stenting any stenosis that is found in any artery of the body.

Thus one object of the present invention is to use the Intro-Angio-Guide catheter for angiography, thus minimizing the opening through the patient's skin.

Another object of the present invention is to use the Intro-Angio-Guide catheter to first perform angiography and then to perform angioplasty and/or stenting using the S-O-A-W stent delivery system, or some other very low profile stent delivery system, thereby providing the smallest possible opening in the skin at the patient's groin.

Another object of this invention is to use an Intro-Angio-Guide system whose outside diameter of its introducer sheath is less than or equal to 5.5 FR to accomplish both angiography and stenting.

Still another object of this invention is to reduce procedure time and lower costs for an interventional procedure by using the same introducer sheath and guiding catheter for both angiography and stenting.

Yet another object of this invention is to construct the introducer sheath component of the Intro-Angio-Guide system using very thin walled tubing that includes either round or flat metal wires that are either braided or helically wrapped within the plastic of the sheath tubing.

Still another object of this invention is to reduce procedure time and lower costs for stenting a blood vessel of the human body by using the Intro-Angio-Guide system with the S-O-A-W system thereby eliminating the need for going to a larger diameter introducer sheath and guiding catheter after angiography reveals the need for stenting, as well as essentially eliminating the need for costly vascular closure devices.

Still another object of this invention to allow the distal portion of the Angio-Guide to be tracked down a coronary artery over the shaft of a balloon angioplasty catheter or a stent delivery system, and thus act as a distal support catheter, to provide “backup” to allow better steering and lesion crossing of the stent system as well as imaging of distal target lesions using a minimum amount of contrast medium.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 illustrates a guiding catheter with a dilator as described in the prior art.

FIG. 2 illustrates the device of FIG. 1 with the dilator removed and the distal section of the catheter having an appropriate curve for entering the ostium of a coronary artery.

FIG. 3A is a side view of the Angio-Guide catheter of the present invention.

FIG. 3B is a side view of the thin-walled introducer sheath of the present invention.

FIG. 3C is a dilator to be used with the Angio-Guide catheter of FIG. 3A.

FIG. 4A shows an assembly view of the present invention with the introducer sheath mounted co-axially around the Angio-Guide catheter with the straightening dilator placed within the shaft of the Angio-Guide catheter and a guide wire placed within the dilator.

FIG. 4B is a side view of the Intro-Angio-Guide catheter system after the dilator and guide wire have been removed.

FIG. 5 is a highly enlarged cross section of the Intro-Angio-Guide catheter at the section “5-5” of FIG. 4B showing the relative thicknesses of the walls of the introducer sheath and the Intro-Angio-Guide catheter and showing the small clearance between the outside surface of the Angio-Guide catheter shaft and the inside surface of the introducer sheath.

FIG. 6 is a longitudinal cross section of the thin-walled introducer sheath placed co-axially around the cylindrical shaft of the Angio-Guide catheter with a very small clearance

FIG. 7A is a longitudinal cross section of the introducer sheath with an inserted sheath dilator and guide wire as configured after the Angio-Guide catheter has been removed from the patient's body.

FIG. 7B is the sectional view of the introducer sheath plus sheath dilator at the section “7B-7B” of FIG. 7A.

FIG. 7C is a longitudinal cross section of the introducer sheath and sheath dilator shown with the sheath dilator snapped onto the proximal end of the introducer sheath.

### DETAILED DESCRIPTION OF THE INVENTION

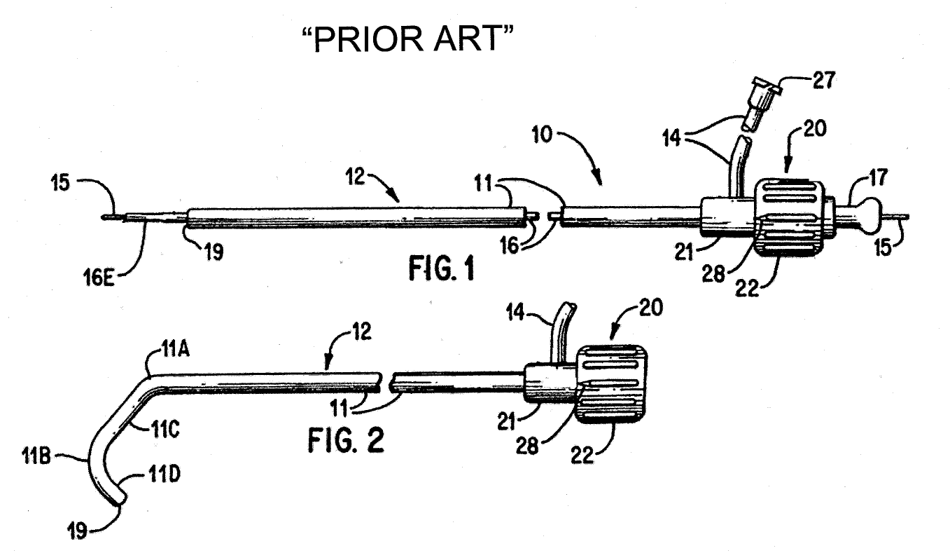

FIGS. 1 and 2 show prior art means for accessing the coronary arteries. In FIG. 1 there is the guiding catheter system 10 consisting of the guiding catheter 12, having a shaft 11 with a distal end 19 and a Touhy-Borst fitting 20 at the proximal end of the shaft 11 which Touhy-Borst fitting 20 has a cylindrical section 22 with grooves 28 and a distal section 21 to which is attached a side arm tube 14 that ends with a Luer fitting 27. Inside the guiding catheter shaft 11 is a dilator 16 having a finger grip proximal end section 17 and a tapered distal section 16E. Within the dilator 16 is a guide wire 15 that is used for advancing the guiding catheter 12 through the groin and into the patient's aorta in close proximity to the heart. The guidewire 15 and dilator 16 are then removed. When that is accomplished, the guiding catheter 12 assumes the shape of its distal section as shown in FIG. 2. This shape has a first bend 11A and a second bend 11B with a straight section 11C being situated between those two curved sections. The end section 11D is designed to be placed through the ostium of a coronary artery to perform angiography or stenting.

The design of FIGS. 1 and 2 would allow angiography and stenting to be performed without the use of an introducer sheath. However, interventional cardiologists generally prefer to have a sheath to remain inserted into the patient's arterial system for some time following the procedure for the injection of medication and to allow repeat access quickly in the case of a sudden reclosure of a coronary artery. It is also preferred to slide the shaft 11 through a sheath (not shown) rather than sliding the entire shaft 11 through both the femoral artery and the opening at the groin.

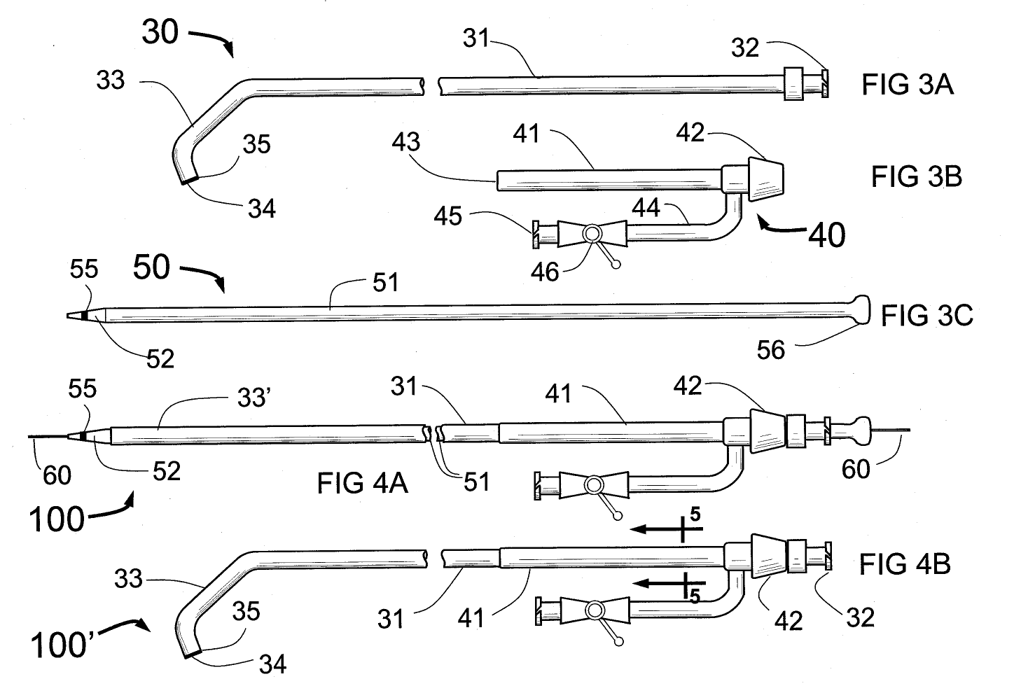

The present invention is designed to accomplish the goal of having the smallest possible opening in the groin while at the same time, having an introducer sheath that can remain in place at the end of the procedure and through which the catheter to access the coronary circulation can slide back and forth as necessary for an angiography or stenting procedure without having a long catheter rubbing the opening in the groin. The invention to accomplish this goal is illustrated in FIGS. 3A to 7C inclusive as presented herein.

FIG. 3A is a side view of an Angio-Guide catheter 30 having a shaft 31, a Luer fitting 32 at its proximal end and a curved distal section 33 having a distal end 34 with radiopaque marker 35. It is well known that there is a need for a variety of curved shapes for the distal section 33 so that the distal end 34 can be placed through the ostium of a coronary artery for patients with various shapes for their aorta in the region of the ostia of the coronary arteries. It is also well known that there are as many as 10 different shapes for the distal section 33 of the Angio-Guide catheter 30 to suite a variety of anatomies.

FIG. 3B shows the thin-walled introducer sheath 40 that is designed to be placed co-axially over the shaft 31 of the Angio-Guide catheter 30. The introducer sheath 40 has a hemostasis valve fitting 42 at its proximal end, a cylindrical shaft 41 with a distal end 43 and a side arm shaft 44 that has a two-way valve 46 near its distal end and a Luer fitting fiting 45 at its distal end.

FIG. 3C shows a dilator 50 having a shaft 51 with a finger grip 56 at the proximal end of the shaft 51 and a tapered distal section 52 with radiopaque marker 55.

FIG. 4A is a side view of the assembled Intro-Angio-Guide catheter 100 showing the introducer sheath shaft 41 co-axially placed around the shaft 31 of the Angio-Guide catheter 30. Within the shaft 31 of the Angio-Guide catheter 30 is the dilator shaft 51 having the tapered distal section 52 with radiopaque marker 55. A guide wire 60 is shown as it is placed through a lumen within the dilator shaft 51. It is in this configuration that the system 100 is placed into the patient's vascular system in order to perform angiography and stenting. The method for doing this is described below.

FIG. 4B is a side view of the Intro-Angio-Guide catheter system 100′ after the guide wire 60 and the dilator 50 have been removed. It is in this configuration that angiography and stenting can be accomplished.

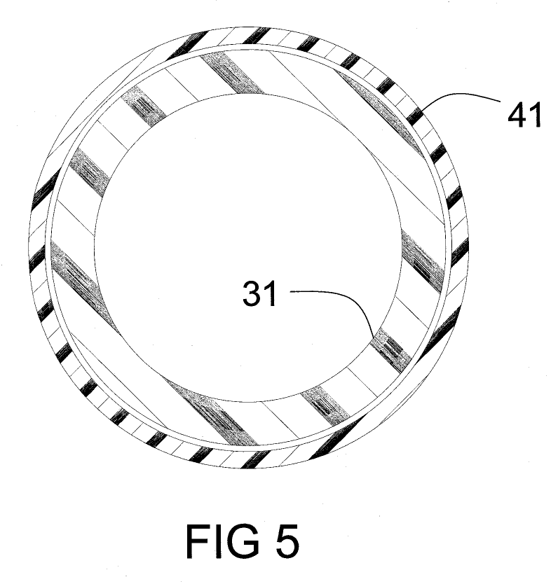

FIG. 5 is a highly enlarged cross section of the Intro-Angio-Guide catheter system 100′ at section “5-5” of FIG. 4B. This cross section illustrates the relative wall thicknesses of the Angio-Guide catheter shaft 31 and the shaft 41 of the introducer sheath 40. A very good design for the Intro-Angio-Guide system 100′ as defined herein would be for the outside diameter of the shaft 31 to be approximately 4.5 FR and the outside diameter of the shaft 41 to be about 5.1 FR As far as dimensions are concerned, in English units, the outside diameter of the shaft 31 would be 59 mils (i.e., 1.5 mm), the clearance between the outside diameter of the shaft 31 and the inside diameter of the shaft 41 would be about 1.0 mil and the wall thickness of the shaft 41 would be about 3 mils. This would provide an outside diameter for the Intro-Angio-Guide system 100′ of approximately 67 mils which is 1.70 mm, and 1.70 mm×3=5.1 FR. It would be a great advance in the fields of both angiography and stenting if one could use an Intro-Angio-Guide catheter 100′ that had an outside diameter of only 5.1 FR for either or both angiography and stenting. For stenting, this compares most favorably to the typical 8 FR or 9 FR (2.7 to 3 mm) outside diameters of introducer sheaths that are currently used for stenting of the coronary arteries. The only reason that this can be accomplished is the novel concept that the very thin-walled sheath shaft 41 will not buckle or collapse only because it is very closely placed around the shaft of the Angio-Guide catheter 30 with a clearance of less than 2 mils and ideally, a clearance of 1.0 mil. All other introducer sheaths sold at this time are sold separately from any angiographic catheter or guiding catheter. Thus, the concept of providing the interventional cardiologist with an integrated catheter system consisting of the thin-walled sheath 40 that is placed by the interventional cardiologist onto the shaft of an angiographic catheter or guiding catheter immediately before insertion into the body of the patient is a completely novel invention and very useful in that the outside diameter of this system is at least 1.5 FR sizes smaller than anything else that is presently available.

To optimize the structural rigidity of the introducer sheath 40, it would be advantageous for the shaft 41 to include some metal wire structure placed within a plastic material such as PTFE or Nylon or any similar plastic material. The metal wire structure (not shown) could be in the form of a wire mesh or a thin-walled, flat wire helix. Stainless steel would be an optimum metal for the wire structure within the shaft 41. The shaft 31 and distal shaft 33 of the Angio-Guide 100 may also include a metal wire structure as is often the case for guiding catheters.

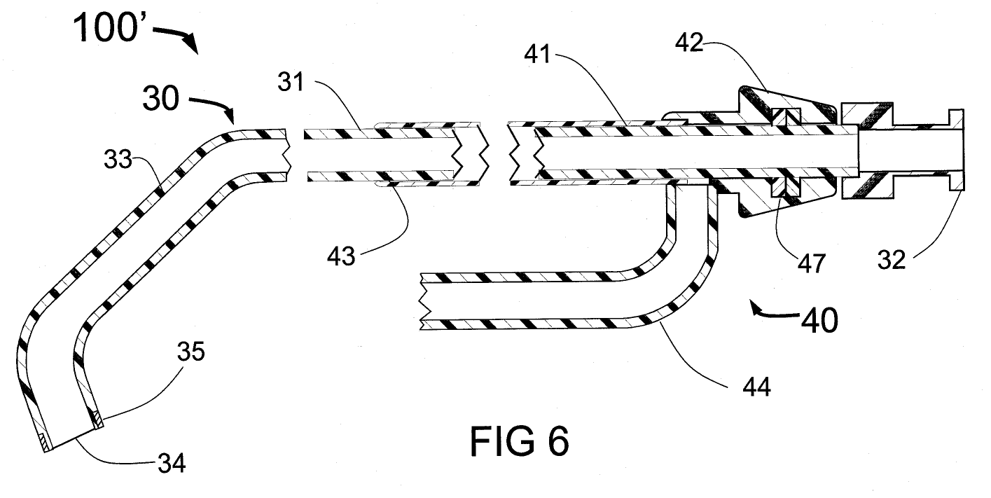

FIG. 6 is a longitudinal cross section of the Intro-Angio-Guide 100′ with the dilator 50 and guide wire 60 removed. In this view it can be seen that wall thickness of the shaft 41 of the introducer sheath 40 is much thinner than the wall thickness of the shaft 31 of the Angio-Guide catheter 30. Also shown in this cross section of the Intro-Angio-Guide 100′ is the cross section of the hemostasis valve 47 within the proximal end fitting 42 of the introducer sheath 40. The function of the hemostasis valve is to prevent leakage of blood from out of the proximal end of the Angio-Guide catheter 30. The Luer fitting 32 at the proximal end of the shaft 31 is typically attached to a Touhy-Borst fitting (not shown) for allowing the passage of a stent delivery system or for injecting medication into the patient while limiting blood loss. It should be understood that the present invention includes the concept of a Touhy-Borst fitting fixedly attached at the proximal end of the Angio-Guide catheter 30 instead of the Luer fitting 32. This design is shown in FIGS. 1 and 2 where the guiding catheter 12 has a Touhy-Borst fitting 20 fixedly attached at the proximal end of the shaft 11.

After the Intro-Angio-Guide 100′ is in place with the distal end 34 of the shaft 31 with radiopaque marker 35 firmly situated through the ostium of a coronary artery, angiography is performed by injecting contrast medium into the heart's arterial system. If no stenosis that warrants revascularization is detected, the angiography procedure is completed and the Intro-Angio-Guide 100′ can be removed from the patient's body in either one or two steps. If there is no reason to leave a sheath for medication or later arterial access, the entire system can be removed. Otherwise, the Angio-Guide catheter 30 is removed and the sheath 40 would remain for a period of time before it too is removed.

Because the outside diameter of the thin-walled introducer sheath 40 is only about 5.1 FR that would be a smaller outside diameter as compared to any other introducer sheath used for angiography that is on the market today. The smallest possible opening in the groin is very important to prevent bleeding at the groin after the introducer sheath is removed.

If the angiography detects a “significant” stenosis in the artery, it is typical in medical practice today to place a stent into that stenosis in order to improve blood flow to the myocardium. If that is the case, then the S-O-A-W stent delivery system as described in U.S. Pat. No. 6,375,660, or other low profile stent delivery system can be used with the Intro-Angio-Guide system 100′ to deliver a stent (not shown) into that stenosis. Because of the very small outside diameter of the S-O-A-W stenting system, stenting can be accomplished without resorting to a larger diameter guiding catheter having a larger diameter introducer sheath. Thus, the combination of the S-O-A-W system with the Intro-Angio-Guide system 100 as described herein would provide by far the smallest opening in the access artery as compared to any other system used for stenting a stenosis. Specifically, instead of an outside diameter of the introducer sheath being 8 FR or 9 FR as is typically used during stenting today, the invention described herein would provide an outside diameter for the introducer sheath 40 that could be as small as approximately 5.1 FR. This may be advantageous in order to avoid the need for vascular closure devices and to reduce the possibility of serious bleeding at the groin which occurs in about 3% of all stent cases today and can result in the patient requiring additional treatments such as a blood transfusion.

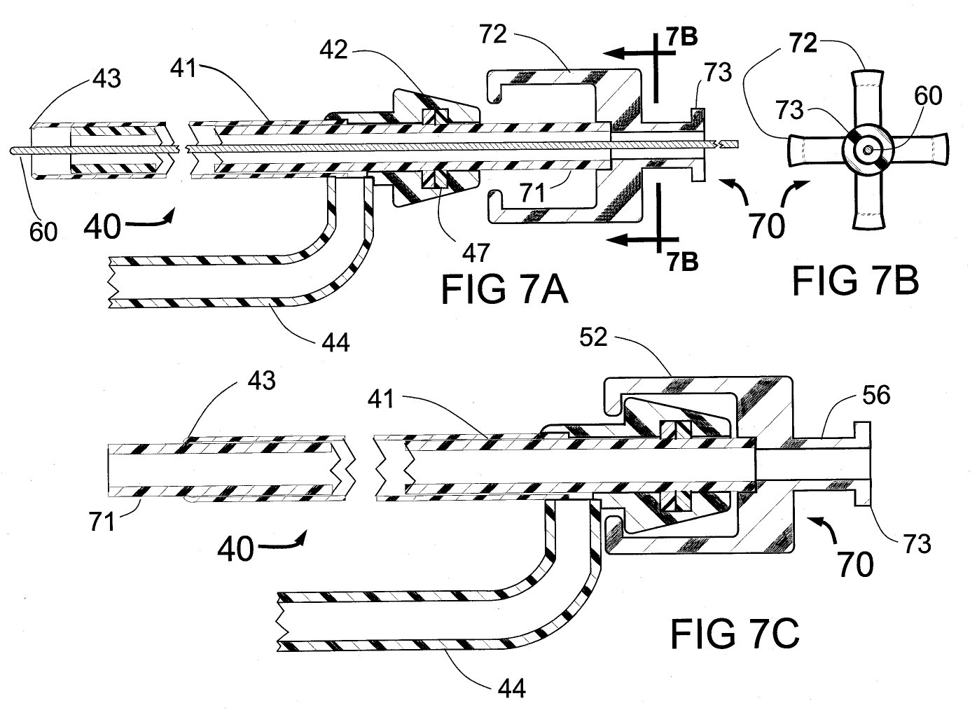

After the angiography or stenting procedure is completed, it is helpful to retain the introducer sheath 40 at its site through the skin at the groin and into the femoral artery so that the patient's medications can be adjusted without significant blood loss through the opening at the groin. For example, medication to prevent blood clots must be mostly gone from the patient's body before the introducer sheath 40 is removed from its site in the groin. Also, the interventional cardiologist may wish to provide additional medications to the patient at the end of the angiography or stenting procedure. For the introducer sheath 40 to remain in the patient's body with the Angio-Guide catheter 30 removed, it is necessary to have a means to prevent the buckling of the thin wall of the shaft 41 of the introducer sheath 40 as the Angio-Guide catheter 30 is withdrawn from the introducer sheath 40. To accomplish this goal, the guide wire 60 (see FIG. 4A) is placed back into the Angio-Guide catheter 30 before the Angio-Guide catheter 30 is removed from the patient's body. After the Angio-Guide catheter 30 is removed, a 35 mil outside diameter guide wire 60 would remain in the shaft 41 of the introducer sheath 40 (see FIG. 7A) to prevent buckling of the shaft 41. The next step in the closure process would be to place a sheath dilator 70 over the guide wire 60 until it snaps onto the proximal end fitting 42 of the introducer sheath 40 as shown in FIG. 7C. This step may not be needed if the thin wall sheath shaft 41 is sufficiently strong to avoid buckling because of the optional wire reinforcement. It will be necessary if the thin wall shaft 41 is not structurally reinforced.

FIG. 7A is a longitudinal cross section of the introducer sheath 40 and the sheath dilator 70 as they would be positioned with the guide wire 60 still in place within the shaft 41 of the introducer sheath 40. FIG. 7B shows the snap-on-arms 72 that are placed at the proximal end of the sheath dilator 70. The purpose of the snap-on-arms 72 is to securely fasten the sheath dilator 70 to the introducer sheath 40 as is shown in FIG. 7C. The proximal end fitting 42 of the introducer sheath 40 has a sloped (actually conical) surface so that the snap-on-arms 72 can be readily moved in a distal direction over the end fitting 42. The configuration shown in FIG. 7C is used until the introducer sheath 40 and the sheath dilator 70 are removed together from the patient's body after the medical staff determines that the patient's condition warrants the removal of these two devices. The Luer fitting 73 at the proximal end of the sheath dilator 70 is used for attaching a mating fitting to prevent blood loss or it can be attached to a means for injecting medication into the patient's vascular system.

Presented below is a detailed method of how the present invention would be used for stenting of a coronary artery.
1. Place a needle into the femoral artery and insert a 35-38 mil guide wire through the needle and into the femoral artery, advancing the wire into the arterial system. Then remove the needle.
2. The Intro-Angio-Guide 100 is then prepared by flushing normal saline between the outside surface of the Intro-Angio-Guide 100 and the inside surface of the shaft 41 of the thin-walled introducer sheath 40 that is pre-mounted onto the Angio-Guide catheter 30. This is accomplished via the side arm 44 of the introducer sheath 40.
3. The Intro-Angio-Guide 100, along with its dilator 50 is then inserted over the guide wire 60 into the target artery (e.g., femoral or radial) and then advanced over the guidewire 60 into the ascending aorta.
4. The dilator 50 and guide wire 60 are then removed from the Intro-Angio-Guide 100, and the Angio-Guide catheter 30 is flushed in a standard fashion.
5. The proximal end of the introducer sheath 40 is then sutured to the patient's skin.
6. The coronary artery (or saphenous vein bypass graft, or internal mammary or other artery to be studied) is then engaged with the distal end 34 of the Angio-Guide catheter 30 being inserted through the ostium of that coronary artery.
7. Angiography is performed in a standard fashion using the Angio-Guide catheter 30 as the diagnostic catheter in order to define the anatomy, and to identify any significant obstructive lesion(s) (i.e., any significant stenosis or stenoses). It is envisioned that different shaped Angio-Guides will be used to engage different target vessels (e.g., left or right coronary arteries).
8. If a significant obstructive lesion is identified, direct stenting can then be performed using the stent-on-a-wire (S-O-A-W) stent delivery system, or other low profile stent delivery system.
9. The S-O-A-W system is advanced through the Angio-Guide catheter 30 and then steered and advanced down the target vessel until the distal end of the S-O-A-W system lies distal to the stenosis to be stented. When necessary the Angio-Guide catheter 30 can be advanced over the body of the S-O-A-W delivery system until its distal end 34 is placed just proximal to the stenosis. In that position, the Angio-Guide catheter 30 acts as both a “sheath” and as a deeply engaged “guiding catheter.”
10. Once the lesion is crossed with the S-O-A-W system, the correct position for placement of the stent is identified using contrast injection(s) via the Angio-Guide catheter 30. Once positioned, the stent on the S-O-A-W system is deployed by inflating the balloon and then deflating the balloon. The deflated S-O-A-W balloon is pulled proximally to allow angiographic study of the stented target lesion. If the result is acceptable the S-O-A-W stenting system is withdrawn and final angiographic images can be taken using the Angio-Guide catheter 30 which is typically pulled back to a more conventional proximal position in the coronary artery (engaged just past the ostium).
11. After the stenosis has been stented, the Angio-Guide catheter 30 is pulled out of the coronary artery and back into the aorta.
12. The guide wire 60 is then placed back into the Angio-Guide catheter 30, and advanced distally past the distal end 34 of the Angio-Guide catheter 30 and into the aorta. The Angio-Guide catheter 30 is then removed from the patient's body, leaving the distal end of the guide wire 60 positioned distally, and lying within the thin-walled introducer sheath 40 in the femoral artery.
13. An optional (short length) sheath dilator 70, whose outside diameter is slightly smaller than the inside diameter of the introducer sheath 40 is then advanced over the mil guide wire 60 with the guide wire's distal end lying distal to the distal end of the introducer sheath 40. The sheath dilator 70 is advanced over the introducer sheath 40 until its snap-on-arms 72 are secured over the proximal end fitting 42 of the introducer sheath 40.
14. The guide wire 60 is then removed from the sheath dilator 70 and the system is “flushed” with saline solution (typically “heparinized” saline).
15. The Luer fitting 73 at the proximal end of the sheath dilator 70 is then attached to a means for injecting fluids and/or medications or monitoring arterial pressure, etc., or it can be closed with a fitting or attached to a three-way stopcock. This is the configuration that is maintained until the patient is able to have the introducer sheath 40 and sheath dilator 70 removed from his/her body which would complete the procedure.

The following would replace steps 13-15 if the sheath 40 includes sufficient wire reinforcement such as a flat wire helix as described in U.S. Pat. No. 5,180,376 that the snap on sheath dilator 70 is not necessary.
13′. The guide wire 60 is then removed from the sheath 40 and the system is “flushed” with saline solution (typically “heparinized” saline).
14′. The Luer fitting 45 of FIG. 3B at the proximal end of the side tube 44 of the sheath 40 is then attached to a means for injecting fluids and/or medications, or it can be closed with a the 2-way valve 46 or attached to a three-way stopcock. This is the configuration that is maintained until the patient is able to have the introducer sheath 40 removed from his/her body which would complete the procedure.

The following procedure could be used if an additional stenosis is to be stented:
1. If the additional stenosis to be stented lies distal to the stented stenosis, or in a side-branch of that same artery, then a second stent delivery system could be used to stent that stenosis, using the same Angio-Guide catheter system 100′.
2. If additional lesions are to be stented in a different target vessel, then most of the steps described above would be repeated except using the correctly shaped Angio-Guide catheter 30 for those stenoses that are accessed through a different target vessels.
3. If there is a proximal or a distal dissection, the Angio-Guide catheter 30 could, in most cases, be advanced over the balloon catheter portion of the S-O-A-W system and advanced through such a dissection. Another S-O-A-W system could then be used to stent the dissection. If the Angio-Guide catheter 30 cannot be placed through the dissection, then a buddy wire technique could be used to place a new coronary guide wire (typically 14 mil diameter) past the dissection. At that point a new guiding catheter could be exchanged using an exchange-length coronary wire, followed by a conventional stenting system to stent the dissection.

The maneuver described in Step 9 above provides three very important performance enhancements compared to conventional stenting with conventional stent delivery systems and guiding catheters: 1) the Angio-Guide catheter 30, when advanced deeply into the target vessel, will provide much better “backup” support and one-to-one torque control and steering of the S-O-A-W system, to allow passage of the S-O-A-W system through a tortuous and/or severely narrowed target lesion (stenosis); 2) contrast can be injected via the Angio-Guide catheter 30 which provides excellent visualization of the target vessel with minimal contrast use (this provides excellent visualization using much less contrast than required using conventional guiding catheters, thereby reducing the risk of contrast-induced nephropathy, volume overload, reduced cost, etc.); and 3) this system allows the Angio-Guide catheter 30 to be advanced over the body of the S-O-A-W delivery system if a dissection occurs during stenting. The S-O-A-W system is advanced distally beyond the dissection, followed by advancement of Angio-Guide over the S-O-A-W to a true lumen position distal to the dissection; allowing removal of S-O-A-W system and replacement with another S-O-A-W system to repair the dissection, and/or a coronary guidewire to be advanced distally to allow conventional stenting and/or replacement of Angio-Guide catheter 30 with a conventional guiding catheter over the coronary guidewire using an exchange length coronary guide wire.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. An integrated catheter system for performing angiography on a human patient, the integrated catheter system consisting of an angiographic catheter onto which a thin-walled sheath is co-axially mounted, the angiographic catheter having an essentially straight and elongated proximal section in the form of a cylindrical shaft that is surrounded for less than one-half of its length by the thin-walled sheath having an outer diameter that is less than 0.25 mm greater than the outside diameter of the angiography catheter, the angiographic catheter being designed to have its proximal section move slideably within the thin-walled sheath.

2. The integrated catheter system of claim 1 where the thin-walled sheath consists of a plastic that is reinforced with metal wire.

3. The integrated catheter system of claim 2 where the metal wire is a stainless steel mesh.

4. The integrated catheter system of claim 2 where the metal wire is a flat wire formed into a helix within the plastic of the sheath.

5. The integrated catheter system of claim 1 where the outside diameter of the thin-walled sheath is less than 1.9 mm.

6. The integrated catheter system of claim 1 where the outside diameter of the thin-walled sheath is approximately 1.7 mm.

7. The integrated catheter system of claim 1 further including a sheath dilator that is designed to be placed into the sheath after the angiographic catheter is removed from the sheath.

8. The integrated catheter system of claim 7 where the sheath dilator has a proximal section that includes snap-on-arms that can be securely attached to a conical portion of the sheath that is located at the sheath's proximal end.

9. The integrated catheter system of claim 1 where the angiographic catheter has a Luer fitting at its proximal end.

10. The integrated catheter system of claim 1 where the angiographic catheter has a Touhy-Borst fitting at its proximal end.

11. An integrated catheter system for performing stenting of a human patient, the integrated catheter system consisting of an angiographic catheter onto which a thin-walled sheath is co-axially mounted and a low profile stent delivery system designed to be advanced through the interior lumen of the angiographic catheter, the angiographic catheter also having an essentially straight and elongated proximal section in the form of a cylindrical shaft that is surrounded for less than one-half of its length by the thin-walled sheath which has a wall thickness of less than 0.13 mm and an outer diameter that is smaller than 1.85 mm, the angiographic catheter being designed to have its proximal section move slideably within the thin-walled sheath.

12. The integrated catheter system of claim 11 where the low profile stent delivery system is the stent-on-a-wire stent delivery system.

13. A method for performing angiography in a human patient, the method including the following steps:
1. placing a thin-walled introducer sheath over the shaft of an angiographic catheter immediately prior to placing that system through a patient's skin at the groin, the clearance between that outside diameter of the angiographic catheter and the inside diameter of the introducer sheath being less than 0.10 mm and the outside diameter of the introducer sheath being less than 1.85 mm, the angiographic catheter having a distal end;
2. advancing the angiographic catheter with the co-axially mounted introducer sheath through an opening in the groin of the human patient and further advancing the angiographic catheter through the access artery and the aorta until its distal end is placed through the ostium of either the left or the right main coronary arteries; and
3. injecting contrast medium through the angiographic catheter to determine if there is a stenosis in one of the coronary arteries.

14. The method of claim 13 further including the step of employing a S-O-A-W stent delivery system, or other very low profile balloon angioplasty catheter or stent delivery system, to treat a stenosis that is found in a coronary artery.

15. The method of claim 13 including the step of placing a S-O-A-W stenting system through a stenosis that is found in a coronary artery and then advancing the distal end of the angiographic catheter to lie just proximal to the stenosis so as to optimize “backup” and the injection of contrast medium.

16. The method of claim 13 further including the step of advancing the angiographic catheter over the wire or the shaft of a stent delivery system, past a dissection of an artery that has been stented and then using another stent delivery catheter to stent the dissection.
